(19) United States
(12) Patent Application Publication
(10) Pub. No.: US 2016/0334694 A1
(43) Pub. Date: Nov. 17, 2016

(54) ENDOSCOPIC DEVICE WITH CLEANING FUNCTION

(71) Applicant: SHENZHEN YONGHENGFENG INTELLIGENT DEVICE CO., LTD, Shenzhen (CN)

(72) Inventors: Zhiguo Liu, Shenzhen (CN); Zhigang Liu, Shenzhen (CN)

(21) Appl. No.: 15/007,137

(22) Filed: Jan. 26, 2016

(30) Foreign Application Priority Data
May 11, 2015 (CN) 201520298533.2
Jul. 9, 2015 (CN) 201520492895.5

(51) Int. Cl. G03B 17/56; A61B 1/00; H04N 7/18; A61B 1/12; H04N 5/225; H04N 5/232

(52) U.S. Cl. CPC G03B 17/568; H04N 5/2256; H04N 5/23209; H04N 7/183; A61B 1/126; A61B 1/00045; H04N 2005/2255

(57) ABSTRACT

An endoscopic device with cleaning function includes an image display, an operating handle, an air pressure tube coupler, a snake tube assembly, a liquid-guiding container, a spray switch, and a liquid-guiding tube; and the snake tube assembly is mounted on the operating handle and wired connected with the image display; the operating handle is provided with a signal transmitting terminal, and the snake tube assembly is wired connected with the signal transmitting terminal; the image display is provided with a signal receiving terminal for wireless signal transmission with the signal transmitting terminal. Under the premise of guaranteeing the acquisition of image functions, the images can be acquired by a wired way of snake tube assembly and image display, or by wireless transmission via the signal transmitting terminal and signal receiving terminal of the snake tube assembly, which can facilitate the cleaning and can check and inspect the surface of objects.

### CROSS-REFERENCE TO RELATED APPLICATIONS

The present application claims the benefit of Chinese Patent Application Nos. 201520492895.5 filed on Jul. 9, 2015 and 201520298533.2 filed on May 11, 2015; the contents of which are hereby incorporated by reference.

### FIELD OF THE INVENTION

This application relates to the endoscopic product field, in particular, to an endoscopic device with cleaning function.

### BACKGROUND OF THE INVENTION

With the social development, the endoscope has been widely applied in the equipment maintenance and metal defect detection, etc. In prior art, for example, China Utility Model Patent (Publication No.: CN203786384U), titled an endoscope connected to a mobile device and endoscope system thereof, disclosed an endoscope connected to a mobile device and endoscope system, and the camera assembly of endoscope is connected with the relay through a metal hose or plastic hose, to facilitate the acquisition of images; however, this kind of endoscope can simply acquire the image information with single function, so it has limitations.

### SUMMARY OF THE INVENTION

In order to overcome the above defects, the application provides an endoscopic device with cleaning functions.

The object of the application is achieved through the following technical solutions:

An endoscopic device with cleaning function comprising an image display, an operating handle, an air pressure tube coupler I, a snake tube assembly, a liquid-guiding container, a spray switch, and a liquid-guiding tube; wherein the image display, air pressure tube coupler I, liquid-guiding container are mounted on the operating handle, and the liquid-guiding tube is arranged on the snake tube assembly; one end of the liquid-guiding tube is mounted on the operating handle and separately connected to liquid-guiding container, air pressure tube coupler I; the spray switch is mounted on the operating handle to control the connection between liquid-guiding tube and liquid-guiding container, air pressure tube coupler I, and the snake tube assembly is mounted on the operating handle and wired connected with the image display;

The operating handle is provided with a signal transmitting terminal, and the snake tube assembly is wired connected with the signal transmitting terminal; the image display is provided with a signal receiving terminal for wireless signal transmission with the signal transmitting terminal.

Further, the image display is provided with a function button set and signal receiving frequency switch, and the operating handle is provided with a signal transmitting frequency switch.

Further, the snake tube assembly comprising: a snake tube, data transmission lines, a front camera, an imaging device; the front camera connected with the imaging device are mounted on the end of the snake tube, the data transmission line is arranged in the snake tube and connected between imaging device and image display, between the image display and the signal transmitting terminal; the snake tube end is also provided with LED lamp connected to data transmission line; the operating handle is also provided with LED control button to control the brightness of LED lamp.

Further, the operating handle comprising: a handle body, an air blow tube, a connector of snake tube body mounted on the handle body, a connector of liquid-guiding tube body, gas valve seat, liquid-guiding container seat used for installing the liquid-guiding container, a suction tube joint, a suction tube, an air pressure tube coupler II, an intake tube, a valve needle, a spring, a seal ring, a pilot valve, and sealing nuts; the end of the snake tube is provided with snake tube fittings, which is connected to the connector of snake tube body; the liquid-guiding tube end is provided with a liquid-guiding tube fittings which is connected to the connector of the liquid-guiding tube body; the suction tube connector is connected to the connector of the liquid-guiding tube body, the suction tube connector is connected to connector of liquid-guiding tube connector, and the suction tube passes through the liquid-guiding container seat to connect the suction tube connector; the intake tube is connected between the air pressure tube coupler I and air pressure tube coupler II, the valve needle is mounted on the gas circuit between the air valve seat and the air pressure tube coupler II; the upper end of the valve needle supports against the spring, and the lower end is sealed by sealing nut, and the middle is coupled with seal ring, pilot valve.

Further, the liquid-guiding tube is fixed with snake tube assembly by clamps of snake tube.

Further, the other end of the liquid-guiding tube is mounted a drainage nozzle.

The application provides an endoscopic device having the cleaning function. Under the premise of guaranteeing the acquisition of image functions, the images can be acquired by a wired way of snake tube assembly and image display, or by wireless transmission via the signal transmitting terminal and signal receiving terminal of the snake tube assembly, which can facilitate the cleaning and spraying operations, such as cleaning pipes, engines, disinfection of objects, leak-proof bonding, etc., besides, it can check and inspect the surface of objects after cleaning and disinfection, leak-proof bonding, with multiple purposes and practicability.

### BRIEF DESCRIPTION OF THE DRAWINGS

For further illustration, the application is described in combination with the following embodiments and drawings.

FIG. 1 shows a schematic view of an endoscopic device with cleaning functions;

FIG. 2 shows a schematic view of image display of an endoscopic device with cleaning functions from a perspective;

FIG. 2 is shows a schematic view of image display of an endoscopic device with cleaning functions from another perspective;

FIG. 4 shows a schematic view of the end part of snake tube assembly in an endoscopic device with cleaning functions;

FIG. 5 shows a schematic view of internal structure of operating handle in an endoscopic device with cleaning functions;

FIG. 6 is a partial enlarged view of FIG.5 for an endoscopic device with cleaning functions.

### DETAILED DESCRIPTION OF THE EMBODIMENTS

In order to further clarify the purpose, technical solutions and advantages of the application, it is further described in details. It should be understood that the specific embodiments described herein are only used to explain the application, not intended to limit it.

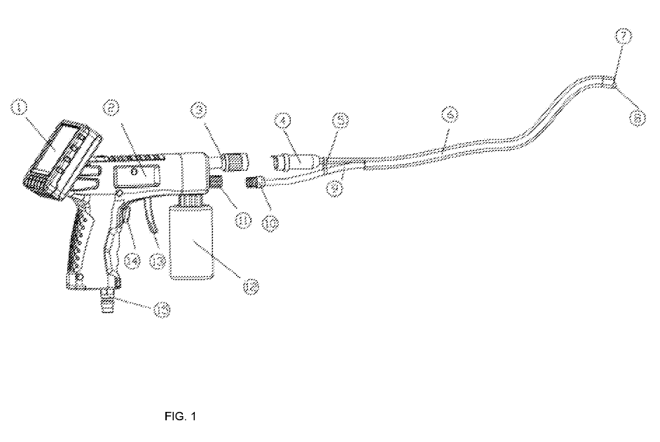

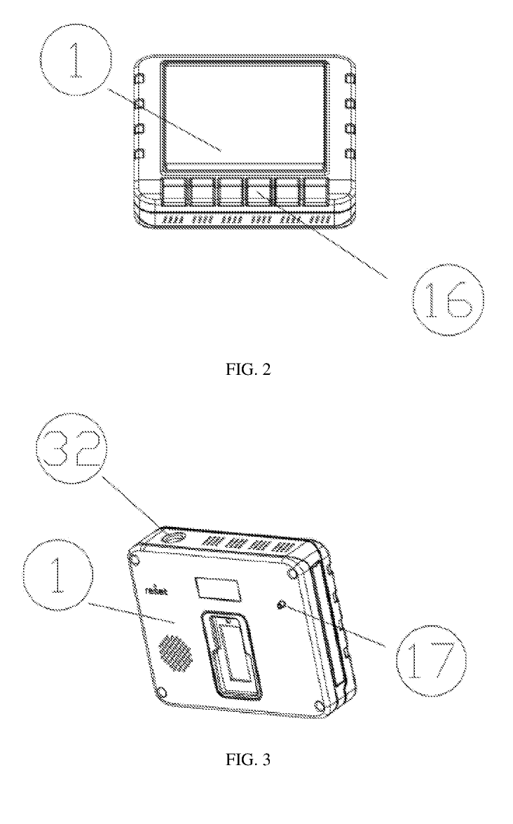

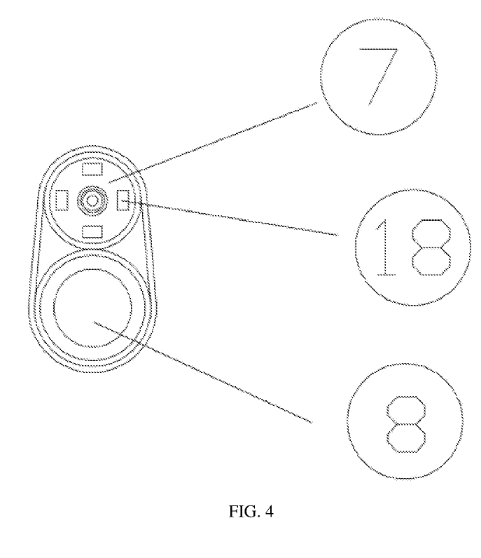

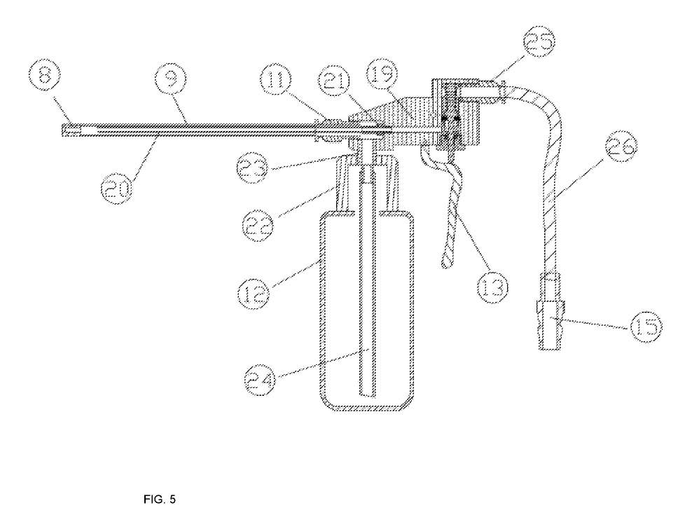

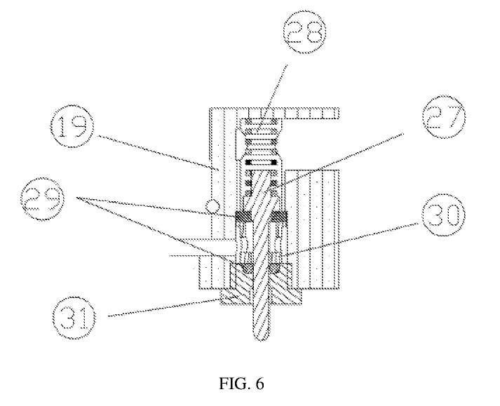

Referring to FIG.1, an endoscopic device with cleaning function comprising an image display 1, an operating handle 2, an air pressure tube coupler I 15, a snake tube assembly 16, a liquid-guiding container 12, a spray switch 13, and a liquid-guiding tube 9; wherein the image display 1, air pressure tube coupler I 15, liquid-guiding container 12 are mounted on the operating handle 2, and the liquid-guiding tube 9 is arranged on the snake tube assembly 6, which is fixed by the snake tube clamp 5; one end of the liquid-guiding tube 9 is mounted on the operating handle 2 and separately connected to liquid-guiding container 12, air pressure tube coupler I 15; and the other end of the liquid-guiding tube 9 is mounted a drainage nozzle 8; the spray switch 13 is mounted on the operating handle 2 to control the connection between liquid-guiding tube 9 and liquid-guiding container 12, air pressure tube coupler I 15.

The snake tube assembly 6 is mounted on the operating handle 2 and wired connected with the image display 1. The operating handle 2 is provided with a signal transmitting terminal (not shown in the figure), and the snake tube assembly 6 is wired connected with the signal transmitting terminal; the image display 1 is provided with a signal receiving terminal for wireless signal transmission with the signal transmitting terminal (not shown in the figure). The signal receiving terminal is a 2.4G Wireless FM signal receiving terminal, which receives the image video sent from the single-band receiving signal transmitting signal under wireless state and multi-screen display on the image screen, e.g. quarter-screen display. Each quarter screen displays the same image video, to facilitate users to check and compare different position of the shoot scenes. The image display 1 is provided with an image display power switch 32, a function button set 16 and signal receiving frequency switch 17, and the operating handle 2 is provided with a signal transmitting frequency switch (not shown in the figure) and an operating handle power switch (not shown in the figure).

The snake tube assembly 6 comprising: a snake tube (not shown in the figure), data transmission lines (not shown in the figure), a front camera 7, an imaging device; the front camera 7 connected with the imaging device are mounted on the end of the snake tube, the data transmission line is arranged in the snake tube and connected between imaging device and image display 1, between the image display and the signal transmitting terminal; the snake tube end is also provided with LED lamp 18 connected to data transmission line; the operating handle 2 is also provided with LED control button 14 to control the brightness of LED lamp.

The operating handle 2 comprising: a handle body 19, an air blow tube 20, a connector 3 of snake tube body mounted on the handle body 19, a connector 11 of liquid-guiding tube body, gas valve seat 22, liquid-guiding container seat 22 used for installing the liquid-guiding container 12, a suction tube joint 23, a suction tube 24, an air pressure tube coupler II 25, an intake tube 26, a valve needle 27, a spring 28, a seal ring 29, a pilot valve 30, and sealing nuts 31; the end of the snake tube is provided with snake tube fittings 4, which is connected to the connector 3 of snake tube body; the liquid-guiding tube end is provided with a liquid-guiding tube fitting 10 which is connected to the connector 11 of the liquid-guiding tube body; the suction tube connector 23 is connected to the connector 11 of the liquid-guiding tube body, and the suction tube 24 passes through the liquid-guiding container seat 22 to connect the suction tube connector 23; the intake tube 26 is connected between the air pressure tube coupler I 15 and air pressure tube coupler II 25, the valve needle 27 is mounted on the gas circuit between the air valve seat 21 and the air pressure tube coupler II 25; the upper end of the valve needle 27 supports against the spring 28, and the lower end is sealed by sealing nut 31, and the middle is coupled with seal ring 29, pilot valve 30. The snake tube assembly 6 is flexibly connected with the operating handle 2 through the matching of snake tube fittings 4 and connector 3 of snake tube body which is easy to disassemble and install. When the front camera 7 is damaged, you just remove and replace it with a new snake tube assembly 6, to facilitate the maintenance and after-sales services. The operating handle 2 can achieve better sealing through the seal ring 29 and sealing nut 31, and the gas pressure is stable and reliable. The valve needle 27 matches with the spring 28; when the valve needle 27 is jacked up by the gas pressure, the valve needle 27 can achieve reset by the elasticity of the spring 28, to guarantee that the gas path is not closed when the gas is not used.

When the endoscopic device with cleaning function can ensure acquisition of image functions, the upper air pressure tube coupler I 15 is externally connected to the high pressure gas, to start the image display 1 and shoot the scene by probing the front camera 7, and adjust the LED control button 14 to obtain a better LED lighting effect. Operate spray switch 13, to carry out surface cleaning on the scene. By observing the images obtained, the cleaning effect can be checked, and the spraying operations, disinfection, leak-proof bonding can be completed, having multiple-purposes; the operating handle 2 and image display 1 are capable of wired and wireless signal transmission; besides, the operating handle 2 and image display 1 have built-in RFID chips, which can burn the delivery date and the electronic data of the each function node of the product. The delivery date can be read via a dedicated scanner, and achieve quickly diagnose the product failures.

The foregoing application has been described in detail by way of illustration and example for purposes of clarity and understanding. As is readily apparent to one skilled in the art, the foregoing are only some of the methods and compositions that illustrate the embodiments of the foregoing invention. It will be apparent to those of ordinary skill in the art that variations, changes, modifications and alterations may be applied to the compositions and/or methods described herein without departing from the true spirit, concept and scope of the application.

What is claimed is:

1. An endoscopic device with cleaning function comprising an image display, an operating handle, an air pressure tube coupler I, a snake tube assembly, a liquid-guiding container, a spray switch, and a liquid-guiding tube; wherein the image display, the air pressure tube coupler I, the liquid-guiding container are mounted on the operating handle, and the liquid-guiding tube is arranged on the snake tube assembly; one end of the liquid-guiding tube is mounted on the operating handle and separately connected to the liquid-guiding container, the air pressure tube coupler I; the spray switch is mounted on the operating handle to control the connection between the liquid-guiding tube and the liquid-guiding container, the air pressure tube coupler I, and the snake tube assembly is mounted on the operating handle and wired connected with the image display;
the operating handle is provided with a signal transmitting terminal, and the snake tube assembly is wired connected with the signal transmitting terminal; the image display is provided with a signal receiving terminal for wireless signal transmission with the signal transmitting terminal.

2. The endoscopic device with cleaning function according to claim 1, wherein the image display is provided with a function button set and signal receiving frequency switch, and the operating handle is provided with a signal transmitting frequency switch.

3. The endoscopic device with cleaning function according to claim 2, wherein the snake tube assembly comprises: a snake tube, data transmission lines, a front camera, an imaging device;
the front camera connected with the imaging device are mounted on the end of the snake tube, the data transmission line is arranged in the snake tube and connected between imaging device and image display, between the image display and the signal transmitting terminal; the snake tube end is also provided with LED lamp connected to data transmission line; the operating handle is also provided with LED control button to control the brightness of LED lamp.

4. The endoscopic device with cleaning function according to claim 3, wherein the operating handle comprises: a handle body, an air blow tube, a connector of snake tube body mounted on the handle body, a connector of liquid-guiding tube body, gas valve seat, liquid-guiding container seat used for installing the liquid-guiding container, a suction tube joint, a suction tube, an air pressure tube coupler II, an intake tube, a valve needle, a spring, a seal ring, a pilot valve, and sealing nuts; the end of the snake tube is provided with snake tube fittings, which is connected to the connector of snake tube body; the liquid-guiding tube end is provided with a liquid-guiding tube fittings which is connected to the connector of the liquid-guiding tube body; the suction tube connector is connected to the connector of the liquid-guiding tube body, the suction tube connector is connected to connector of liquid-guiding tube connector, and the suction tube passes through the liquid-guiding container seat to connect the suction tube connector; the intake tube is connected between the air pressure tube coupler I and air pressure tube coupler II, the valve needle is mounted on the gas circuit between the air valve seat and the air pressure tube coupler II; the upper end of the valve needle supports against the spring, and the lower end is sealed by sealing nut, and the middle is coupled with seal ring, pilot valve.

5. The endoscopic device with cleaning function according to claim 4, wherein the liquid-guiding tube is fixed with snake tube assembly by clamps of snake tube.

6. The endoscopic device with cleaning function according to claim 4, wherein the other end of the liquid-guiding tube is mounted a drainage nozzle.
